(19) United States
(12) Patent Application Publication
(10) Pub. No.: US 2011/0039902 A1
(43) Pub. Date: Feb. 17, 2011

(54) PROCESS FOR PREPARING A CRYSTALLINE FORM COMPOUND OF 3-BENZYL-2-METHYL-2,3,3a,4,5,6,7,7a-OCTAHYDROBENZO[d]ISOXAZOL-4-ONE

(75) Inventors: Fabio Neggiani, Pisa (IT); Elio Napolitano, Pisa (IT); Simone Basagni, Pisa (IT); Barbara Politi, Livorno (IT)

Correspondence Address: Steinfl & Bruno, 301 N Lake Ave Ste 810, Pasadena, CA, 91101, US

(73) Assignee: ABIOGEN PHARMA S.P.A., Ospedaletto (IT)

(21) Appl. No.: 12/989,069

(22) PCT Filed: Apr. 23, 2009
(86) PCT No.: PCT/EP09/54859

§ 371 Date: Oct. 21, 2010

(30) Foreign Application Priority Data
Apr. 24, 2008 (IT) MI2008A000768

(51) Int. Cl. A61K 31/423; C07D 261/20; A61P 25/22; A61P 25/24; A61P 25/08; A61P 25/30

(52) U.S. Cl. 514/379; 548/241

(57) ABSTRACT

A process for preparing a crystalline form compound of 3-benzyl-2-methyl-2,3,3a,4,5,6,7,7a-octahydrobenzo[d]isoxazol-4-one is described, comprising the step of reacting 3-benzyl-2-methyl-2,3,3a,4,5,6,7,7a-octahydroben-zo[d]isoxazol-4-one with oxalic acid in one or more solvents, wherein at least one solvent is a solvent having a carbon atom number of from 3 to 6, said solvent being non-halogenated and having a dielectric constant ∈ within the range from 4 to 25. The process of the invention enables a co-crystal comprising 3-benzyl-2-methyl-2,3,3a,4,5,6,7,7a-octahydrobenzo[d]isoxazol-4-one and oxalic acid to be obtained.

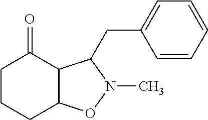

### FIELD OF THE INVENTION

The present invention concerns a process for preparing a crystalline form compound of 3-benzyl-2-methyl-2,3,3a,4,5,6,7,7a-octahydrobenzo[d]isoxazol-4-one of formula

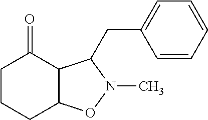

in all its stereochemical configurations, a co-crystal obtained by said process comprising 3-benzyl-2-methyl-2,3,3a,4,5,6,7,7a-octahydrobenzo[d]isoxazol-4-one and oxalic acid, and its use for the treatment of mood disorders, anxiety disorders, depression, convulsive states, in the improvement of learning ability, in the reversal of amnesia, in resolving abstinence syndrome from medicaments and drugs.

### STATE OF THE ART

The compound rel-(3R,3aS,7aS)-3-benzyl-2-methyl-2,3,3a,4,5,6,7,7a-octahydrobenzo[d]isoxazol-4-one, also known as BTG 1640, is described in international patent application WO93/17004 and pertains to a new family of psychoactive agents.

According to this document, the BTG 1640 compound is prepared as a yellow oil then salified as a hydrochloride salt.

Said preparation, which comprises use of the oily free base to form the hydrochloride salt, requires a complicated crystallization and purification step to obtain a pharmaceutical grade salt.

Thus, there is still a need for a BTG 1640 compound in crystalline form which is easy to prepare, that is to say not requiring difficult and lengthy crystallizations and can hence be easily scaled-up to industrial levels.

The object of the present invention is therefore to provide the compound BTG 1640 in crystalline form which responds to the need for an industrially scalable process.

### SUMMARY

As the hydrochloride salt of BTG 1640 presented not only preparation problems, but was also unstable at temperatures above 30° C., the inventors of the present invention have turned their attention to the preparation of compounds alternative to BTG 1640 hydrochloride.

The aforementioned object was hence achieved through the selection of oxalic acid and the selection of reaction solvent(s).

The invention therefore concerns a process for preparing a crystalline form compound of 3-benzyl-2-methyl-2,3,3a,4,5,6,7,7a-octahydrobenzo[d]isoxazol-4-one which comprises the step of reacting 3-benzyl-2-methyl-2,3,3a,4,5,6,7,7a-octahydrobenzo[d]isoxazol-4-one with oxalic acid in one or more solvents, wherein at least one solvent is a solvent having a carbon atom number of from 3 to 6, said solvent being non-halogenated and having a dielectric constant ∈ in the range from 4 to 25.

In a preferred aspect, the invention concerns a process for preparing a crystalline form compound of 3-benzyl-2-methyl-2,3,3a,4,5,6,7,7a-octahydrobenzo[d]isoxazol-4-one which comprises the step of reacting 3-benzyl-2-methyl-2,3,3a,4,5,6,7,7a-octahydrobenzo[d]isoxazol-4-one with oxalic acid in a 2:1 molar ratio, in one or more solvents, wherein at least one solvent is a solvent having a number of carbon atoms from 3 to 6, said solvent being non-halogenated and having a dielectric constant ∈ in the range from 4 to 25.

The inventors of the present invention have surprisingly found that the crystalline form BTG 1640 compound obtained by the process of the invention is a co-crystal. Another aspect of the invention, therefore, concerns a co-crystal comprising 3-benzyl-2-methyl-2,3,3a,4,5,6,7,7a-octahydrobenzo[d]isoxazol-4-one and oxalic acid.

In a further aspect of the invention the co-crystal comprising 3-benzyl-2-methyl-2,3,3a,4,5,6,7,7a-octahydrobenzo[d]isoxazol-4-one and oxalic acid is used as a medicament.

In yet a further aspect of the invention, the co-crystal comprising 3-benzyl-2-methyl-2,3,3a,4,5,6,7,7a-octahydrobenzo[d]isoxazol-4-one and oxalic acid is used for the production of a medicament for treating mood disorders, anxiety disorders, depression, convulsive states, in the improvement of learning ability, in the reversal of amnesia, in resolving abstinence syndrome from medicaments and drugs.

### DESCRIPTION OF THE FIGURES

The characteristics and advantages of the invention will be evident from the detailed description that follows and from the accompanying figures, in which:

FIG. 1 shows the observed experimental x-ray powder diffractogram of the 3-benzyl-2-methyl-2,3,3a,4,5,6,7,7a-octahydrobenzo[d]isoxazol-4-one and oxalic acid co-crystal of the invention;

FIG. 2 shows the experimental diffractogram calculated from information obtained from X-ray diffractography on a single crystal of the 3-benzyl-2-methyl-2,3,3a,4,5,6,7,7a-octahydrobenzo[d]isoxazol-4-one and oxalic acid co-crystal of the invention; and

FIG. 3 shows the table containing a list of the characteristic peaks of the calculated experimental diffractogram shown in FIG. 2.

### DETAILED DESCRIPTION OF THE INVENTION

The invention therefore concerns a process for preparing a crystalline form compound of 3-benzyl-2-methyl-2,3,3a,4,5,6,7,7a-octahydrobenzo[d]isoxazol-4-one which comprises the step of reacting 3-benzyl-2-methyl-2,3,3a,4,5,6,7,7a-octahydrobenzo[d]isoxazol-4-one with oxalic acid in one or more solvents, wherein at least one solvent is a solvent having a number of carbon atoms of from 3 to 6, said solvent being non-halogenated and having a dielectric constant ∈ in the range from 4 to 25.

The reaction between 3-benzyl-2-methyl-2,3,3a,4,5,6,7,7a-octahydrobenzo[d]isoxazol-4-one and oxalic acid takes place in the presence of one or more solvents. In this respect, the inventors of the present invention perceived they had obtained a compound in crystalline form by means of a process suitable for industrial scale-up, through the selection of at least one reaction solvent according to specific physical and structural characteristics.

Said reaction solvent is therefore a non-halogenated solvent, having a number of carbon atoms from 3 to 6 and having a dielectric constant ∈ in the range from 4 to 25.

The at least one solvent of the invention is preferably selected from the group consisting of diethylether, t-butyl-methyl-ether (MTBE), 1,2-dimethoxyethane, tetrahydrofuran (THF), diisopropylether, 4-methyl-2-pentanone, 2-methoxyethanol, 2-butanol, 2-methyl-1-propanol, 2-propanol, 2-butanone, 1-propanol, 1-butanol, acetone.

In the most preferred embodiment of the invention the at least one reaction solvent is chosen from the group consisting of t-butyl-methyl-ether (MTBE), 1-butanol, acetone. In said embodiment, advantageously, the yield of the crystalline form compound is in the range from 80% to 99%.

Advantageously, by using at least one solvent of the invention chosen from the group consisting of t-butyl-methyl-ether (MTBE), 1-butanol and acetone, the reaction between BTG 1640 and oxalic acid can be conducted with decidedly moderate amounts of solvents, i.e. a ratio of solvent quantity/mmol of reacting oxalic acid within the range from 2 to 10, and preferably within the range from 2.5 to 5.

In a preferred aspect, the invention concerns a process for preparing a crystalline form compound of 3-benzyl-2-methyl-2,3,3a,4,5,6,7,7a-octahydrobenzo[d]isoxazol-4-one which comprises the step of reacting 3-benzyl-2-methyl-2,3,3a,4,5,6,7,7a-octahydrobenzo[d]isoxazol-4-one with oxalic acid in a 2:1 molar ratio, in one or more solvents, wherein at least one solvent is a solvent having a number of carbon atoms of from 3 to 6, said solvent being non-halogenated and having a dielectric constant ∈ in the range from 4 to 25.

In the process of the invention oxalic acid is hence reacted with the BTG 1640 free base, preferably in a 1:2 ratio.

Preferably in the process of the invention oxalic acid is added under reflux to the solution consisting of the BTG 1640 free base dissolved in one or more solvents, said reflux conditions being maintained until a clear solution is obtained. The solution is then cooled to a temperature in the range from room temperature to −25° C. for a time varying from 2 to 24 hours.

Advantageously, if the at least one reaction solvent is acetone or 1-butanol, a clear solution of the two reagents BTG 1640 and oxalic acid can be obtained while under agitation even at room temperature, without needing to reach solution reflux temperature.

Advantageously, if the at least one reaction solvent is methyl-t-butyl-ether (MTBE) or acetone, yields exceeding 80% can already be obtained by cooling the solution of the two reagents to ambient temperature.

The compound in crystalline form which separates as a colourless compound can be optionally subjected to further purification cycles according to the known art. The mother liquors can possibly be evaporated and subjected to prolonged cooling with the aim of recovering more of the BTG 1640 and oxalic acid compound in crystalline form.

The compound in crystalline form which separates from the process of the invention is a co-crystal comprising oxalic acid and BTG 1640 free base.

As will be demonstrated in the experimental part, the co-crystal of the invention is a crystalline molecular complex, i.e. a combination of the two molecules BTG 1640 and oxalic acid spatially disposed to create a single crystal form.

The co-crystal of the invention was characterized by X-ray diffractometry carried out on both the powders and the single crystal.

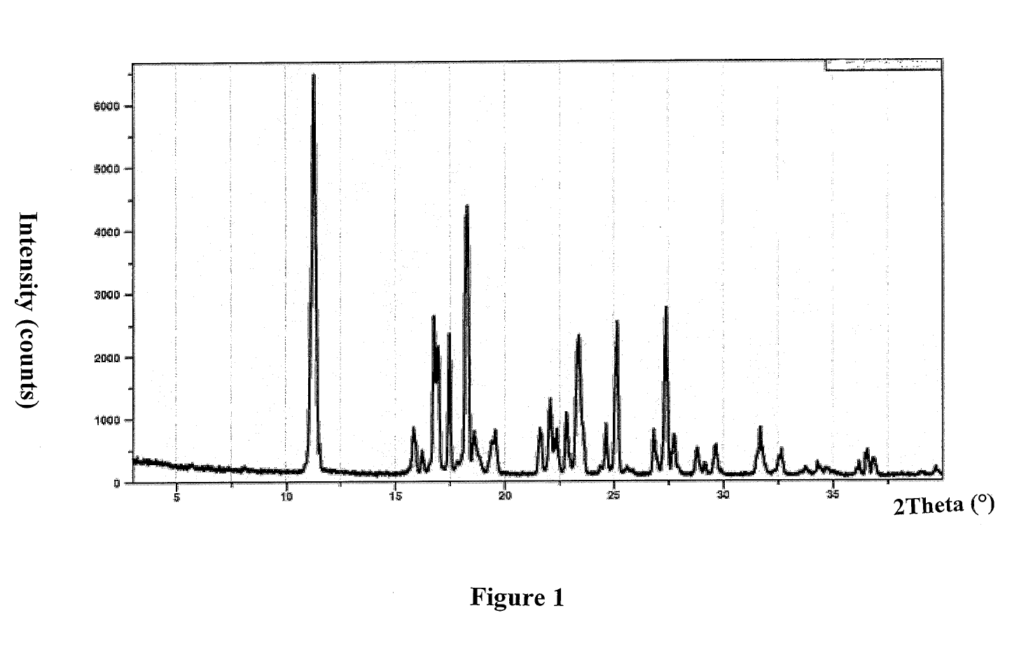

X-ray powder diffractometry was used to obtain the observed experimental diffractogram shown in FIG. 1, from which it is inferred that the co-crystal of the invention exhibits peaks at the diffraction degrees (±0.2° 2θ) shown in Table 1 below:

TABLE 1

8.15
11.26
15.80
16.19
16.74
16.96
17.46
17.84
18.31
18.59
19.38
19.56
21.52
21.67
22.06
22.36
22.80
23.39
24.62
25.14
25.59
26.79
27.38
27.73
28.80
29.11
29.64
31.67
32.64
33.75
34.28
34.70
36.17
36.47
36.57
36.80
39.03
39.66

More specifically, the diffractogram relating to the co-crystal of the invention exhibits characteristic peaks at the following diffractometer angles:

11.26° 2θ
15.80° 2θ
17.46° 2θ
18.31° 2θ
19.55° 2θ
24.62° 2θ

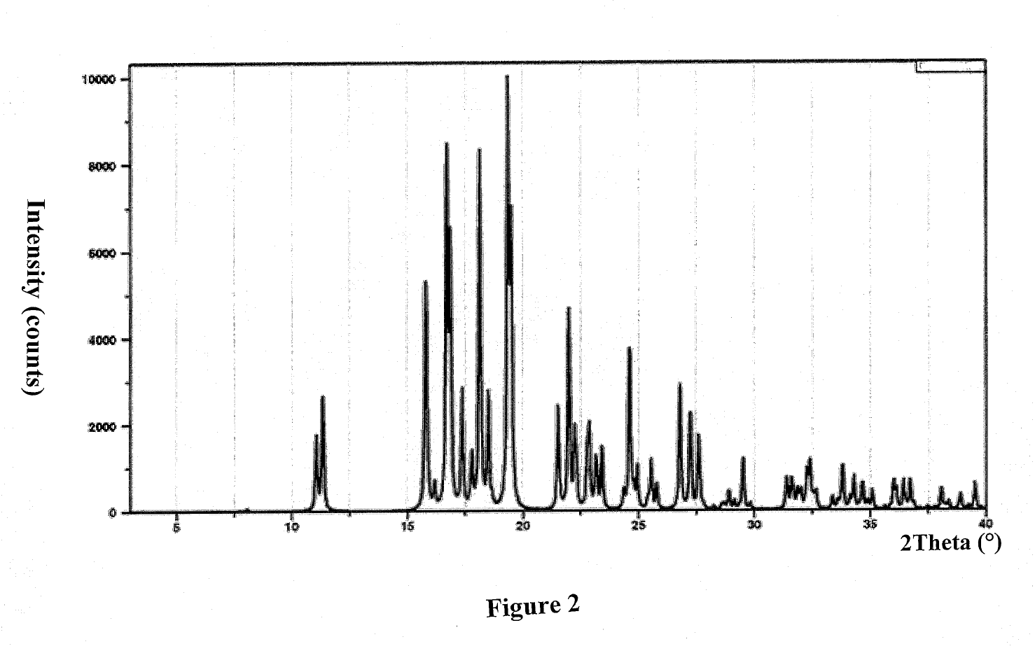

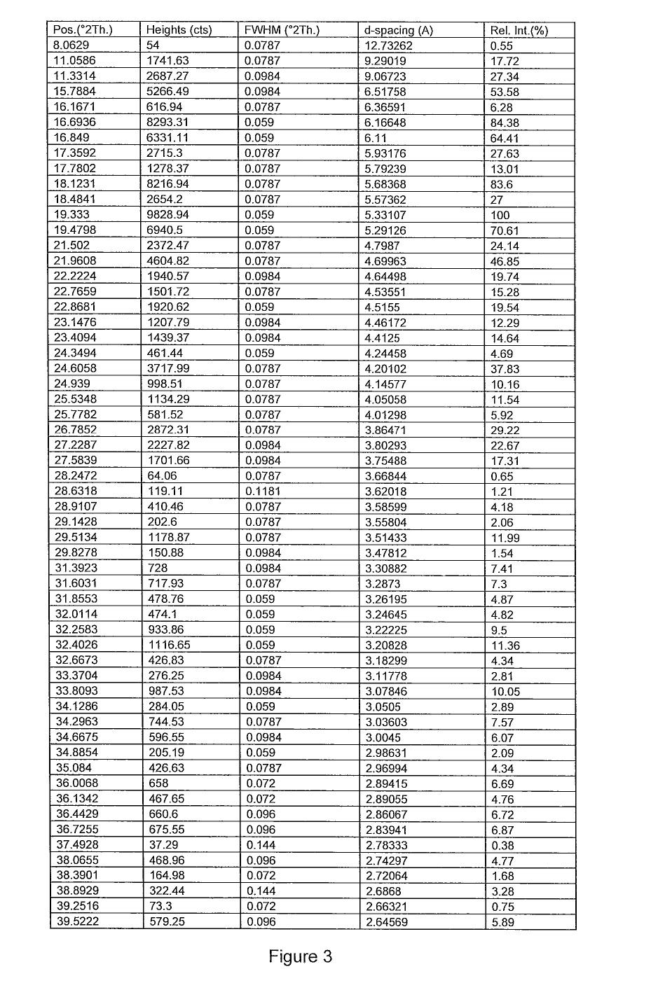

Single crystal X-ray diffractometry was used to obtain information on the structure and on the interatomic distances of the molecules involved, which confirmed the fact that the crystalline solid is a co-crystal consisting of two molecules of BTG 1640 and one of oxalic acid. Calculations on the data obtained from the single crystal diffraction analysis on the BTG 1640 and oxalic acid co-crystal, have generated the calculated experimental diffractogram, which is devoid of any of the imperfections and background noise typical of microcrystalline powders. The calculated experimental diffractogram is given in FIG. 2, the characteristic peaks of which are given in the subsequent FIG. 3.

According to the invention, the co-crystal is therefore obtained by a simple procedure, easily scalable to industrial levels and avoiding the use of lengthy and costly crystallization and purification steps, to obtain high yields of a pharmaceutical grade stable crystalline form.

The co-crystal comprising BTG 1640 and oxalic acid of the invention can be used as a medicament.

It can then be combined with a pharmaceutically acceptable carrier and, optionally, with suitable excipients, to obtain pharmaceutical compositions. The term “pharmaceutically acceptable carrier” includes solvents, diluents and the like which are used in the administration of the co-crystals of the invention.

Said pharmaceutical compositions can be parenterally, orally or topically administered.

Compositions of the present invention suitable for oral administration will be conveniently in the form of discrete units such as tablets, capsules, cachets, as powders or granules, or as a suspension in a liquid.

More preferably the compositions of the invention for oral administration will be in the form of tablets.

The tablets will preferably comprise an amount from 1 to 100 mg, even more preferably from 1 to 50 mg, of the co-crystal comprising oxalic acid and BTG 1640. Preferably the tablets will contain from 1.7% to 40% by weight of the co-crystal comprising BTG 1640 and oxalic acid, and even more preferably the co-crystal comprising BTG 1640 and oxalic acid will constitute from 2.1% to 34.7% of the total tablet weight.

The tablets could also contain suitable excipients in common pharmaceutical use such as pre-gelatinized starch, microcrystalline cellulose, sodium starch glycolate, talc, lactose, magnesium stearate, sucrose, stearic acid, mannitol.

Compositions for parenteral administration will conveniently comprise sterile preparations.

Preparations for parenteral administration will preferably comprise an amount from 0.1 to 100 mg of co-crystal comprising oxalic acid and BTG 1640.

Compositions for topical administration will be conveniently in the form of creams, pastes, poultices, oils, ointments, emulsions, foams, gels, drops, aqueous solutions, spray solutions and transdermal patches.

Preparations for topical administration will preferably comprise an amount from 1 to 100 mg of co-crystal comprising oxalic acid and BTG 1640.

The co-crystal of the invention can be used for the production of a medicament for the treatment of mood disorders, anxiety disorders, depression, convulsive states, in the improvement of learning ability, in the reversal of amnesia, in resolving abstinence syndrome from medicaments and drugs.

The invention will now be described in greater detail in the following examples, given by way of non-limiting illustration of the invention, relative to the process of the invention and to the characterization of the co-crystal obtained by the process.

### EXAMPLES

### Example 1

### Process for Preparing BTG 1640 and Oxalic Acid Co-Crystal in Acetone

0.8079 g of BTG 1640 free base (3.29 mmol) were placed in a 25 ml flask containing 5 ml of acetone; 0.148 g (1.64 mmol) of anhydrous oxalic acid were added at room temperature to the thus obtained solution to obtain a still clear solution.

From the solution kept at ambient temperature for 2 hours, the BTG 1640 and oxalic acid co-crystal (Tmelting from 127 to 130° C.) was separated with a yield of 92%.

### Example 2

### Process for Preparing the BTG 1640 and Oxalic Acid Co-Crystal in Tert-Butyl-Methyl-ether (MTBE)

0.8107 g of BTG 1640 free base (3.30 mmol) were placed in a 25 ml flask containing 5 ml of tert-butyl-methyl-ether (MTBE); 0.149 g (1.65 mmol) of anhydrous oxalic acid were then added at reflux to the solution thus obtained. Reflux conditions were maintained until a clear solution was obtained. From the solution cooled for 4 hours to ambient temperature, the BTG 1640 and oxalic acid co-crystal (Tmelting between 127 and 130° C.) was separated with a yield of 82%.

### Example 3

### Process for Preparing the BTG 1640 and Oxalic Acid Co-Crystal in 1-Butanol

0.801 g of BTG 1640 free base (3.26 mmol) were placed in a 25 ml flask containing 5 ml of 1-butanol; 0.147 g (1.63 mmol) of anhydrous oxalic acid were then added at room temperature to the thus obtained solution to obtain a still clear solution. From the solution cooled for 15 hours to a temperature of 4° C., the BTG 1640 and oxalic acid co-crystal (Tmelting between 127 and 130° C.) was separated with a yield of 95%.

In particular from examples 1-3 the authors of the present invention have found that excellent yields of the BTG 1640 and oxalic acid co-crystal were obtained by mixing BTG 1640 and oxalic acid in a molar ratio of 2:1. These operating conditions enable reagent wastage, usually accompanying reactions not conducted under stoichiometric conditions, to be avoided, and to especially avoid the problem of managing the excesses which should in any case be removed from the reaction medium, and possibly recovered or reconverted.

### Example 4

### Characterization of the Co-Crystal by Means of X-Ray Diffractometry on the Single Crystal

The co-crystal obtained in example 1 was analysed to determine its structure. Specifically, a colourless needle crystal of the co-crystal of example 1, being 0.2×0.2×0.3 mm in size, was mounted onto a glass fibre in a random orientation. The crystallographic data were collected at room temperature using a Nonius CAD-4 diffractometer, Mo-κα radiation, α=0.71073 Å, with a graphite monochromator.

The cell parameters and an orientation matrix for data collection were obtained by the least-squares method using the setting angles of 25 reflections within the range 7°<θ<15°.

The space group was determined by means of the XPREP programme. The space group was P21/n. The structure was solved by direct methods and refined using the full-matrix least-squares method on F² with the SHELX-97 programme.

The crystallographic data obtained are summarized in Table 2 below.

TABLE 2
Crystallographic data of the compound of the invention in crystalline form.

| | |
|---|---|
| Empirical Formula | C₃₂H₄₀N₂O₈ |
| Formula weight | 580.68 |
| Temperature | 20° C. |
| Wavelength | 0.71073 Å |
| Space group | Monocline, P21/n |
| Unit cell dimensions | a = 15.6171(6) Å alpha = 90° |
| | b = 5.9951(3) Å beta = 92.233(4)° |
| | c = 15.9995(5) Å gamma = 90° |
| Volume | 1496.83(11) A³ |
| Z value, calculated density | 4, 1.275 Mg/m³ |
| Absorption coefficient | 0.087 mm⁻¹ |
| F(000) | 616 |
| Crystal dimensions | 0.25 × 0.20 × 0.18 mm |
| Theta range for data | 2.55-25.00° C. |
| collection | |
| limiting indices | −17 ≦ h ≦ 18, −7 ≦ k ≦ 7, −19 ≦ l ≦ 19 |
| Reflections | 11706/2628 [R(int) = 0.0221] |
| collected/unique | |
| Completeness to theta | 25.00 99.8% |
| Refinement method | full-matrix least-squares on F² |
| Data/restraints/parameters | 2628/0/204 |
| Goodness of fit on F² | 1.084 |
| Final R indices | R1 = 0.0859, wR2 = 0.2513 |
| [I > 2sigma(I)] | |
| R indices (all data) | R1 = 0.1025, wR2 = 0.2664 |
| Extinction coefficient | 0.024 (8) |
| Largest diffractometric | 0.965 e-0.692 eA⁻³ |
| peak and hole | |

The results of the crystallographic analysis have demonstrated that in samples of the substance obtained according to example 1, there is no transfer of hydrogen between the oxalic acid carboxyl groups and the nitrogen atom of the BTG 1640 free base confirming that the compound of the invention is a co-crystal of oxalic acid and BTG 1640.

FIGS. 2 and 3 show respectively the calculated experimental diffractogram for the co-crystal and the corresponding table listing the values of the various peaks in said diffractogram.

### Example 5

### Co-Crystal Characterization by Determination of X-Ray Powder Diffractogram

An X-ray powder diffractogram was obtained from the same sample as example 4 by the X'Pert Pro analytical automated diffractometer equipped with X'Celerator, Cuκα, using glass sample holders and 150 mg of substance, setting the voltage and amperage to 40 kV and 40 mA respectively. The data collection programme used was set to obtain the data within the 2 theta range from 3° to 40°.

The observed experimental diffractogram is shown in FIG. 1.

The peaks are given in table 1 below:

TABLE 1
8.15
11.26
15.80
16.19
16.74
16.96
17.46
17.84
18.31
18.59
19.38
19.56
21.52
21.67
22.06
22.36
22.80
23.39
24.62
25.14
25.59
26.79
27.38
27.73
28.80
29.11
29.64
31.67
32.64
33.75
34.28
34.70
36.17
36.47
36.57
36.80
39.03
39.66

### Example 6

The same analyses as in examples 4 and 5 were carried out on the samples obtained from preparative examples 2-3. The results obtained are in agreement with the results given in examples 4 and 5, confirming the fact that all the preparative conditions of examples 2-3 have led to the BTG 1640 and oxalic acid co-crystal of the invention being obtained.

The process of the invention, being simple and of immediate industrial scale-up, has hence provided a new crystalline form which is a co-crystal comprising BTG 1640 and oxalic acid.

1. A process for preparing a crystalline form compound of 3-benzyl-2-methyl-2,3,3a,4,5,6,7,7a-octahydrobenzo[d]isoxazol-4-one, the process comprising the step of
reacting 3-benzyl-2-methyl-2,3,3a,4,5,6,7,7a-octahydrobenzo[d]isoxazol-4-one with oxalic acid in one or more solvents,
wherein at least one solvent of the one or more solvents is a solvent having a carbon atom number of from 3 to 6, said solvent being non-halogenated and having a dielectric constant ∈ in the range from 4 to 25.

2. The process according to claim 1, wherein the 3-benzyl-2-methyl-2,3,3a,4,5,6,7,7a-octahydrobenzo[d]isoxazol-4-one is reacted with oxalic acid in a molar ratio of about 2:1.

3. The process according to claim 1, wherein the at least one solvent is chosen from the group consisting of diethylether, t-butyl-methyl-ether (MTBE), 1,2-dimethoxyethane, tetrahydrofuran (THF), diisopropylether, 4-methyl-2-pentanone, 2-methoxyethanol, 2-butanol, 2-methyl-1-propanol, 2-propanol, 2-butanone, 1-propanol, 1-butanol, and acetone.

4. The process according to claim 3, wherein the at least one solvent is chosen from the group consisting of t-butyl-methyl-ether (MTBE), 1-butanol, and acetone.

5. The process according to claim 4, wherein the reacting provides a yield of the crystalline form compound, and the yield of the crystalline form compound is in a range 80% to 99%.

6. The process according to claim 4, wherein the reaction between 3-benzyl-2-methyl-2,3,3a,4,5,6,7,7a-octahydrobenzo[d]isoxazol-4-one and oxalic acid is conducted with a solvent volume/mmol of oxalic acid ratio within a range from 2 to 10.

7. The process according to claim 1, wherein the reacting comprises adding oxalic acid at solvent reflux conditions until a clear solution is obtained.

8. The process according to claim 4, wherein the at least one reaction solvent is acetone or 1-butanol, and the reacting comprises adding oxalic acid under agitation at room temperature until a clear solution is obtained.

9. The process according to claim 1, wherein the reacting comprises cooling a solution containing the oxalic acid and the 3-benzyl-2-methyl-2,3,3a,4,5,6,7,7a-octahydrobenzo[d]isoxazol-4-one at a temperature within a range from room temperature to −25° C. for a time varying from 2 to 24 hours.

10. The process according to claim 4, wherein the at least one reaction solvent is acetone or methyl-t-butyl-ether (MTBE), the reacting provides a yield of the crystalline form compound, and the yield already exceeds 80% by cooling the reaction to one or more temperatures, the one or more temperatures comprising medium to ambient temperature.

11. A co-crystal comprising oxalic acid and 3-benzyl-2-methyl-2,3,3a,4,5,6,7,7a-octahydrobenzo[d]isoxazol-4-one.

12. The co-crystal according to claim 11, wherein the co-crystal exhibits an X-ray powder diffractogram having characteristic peaks expressed in diffraction degrees (±0.2° 2θ) at 11.26, 15.80, 17.46, 18.31, 19.55 and 24.62±0.2° 2θ.

13. The co-crystal according to claim 11, wherein the co-crystal exhibits an X-ray powder diffractogram having the following peaks expressed in diffraction degrees (±0.2° 2Θ):
8.15
11.26
15.80
16.19
16.74
16.96
17.46
17.84
18.31
18.59
19.38
19.56
21.52
21.67
22.06
22.36
22.80
23.39
24.62
25.14
25.59
26.79
27.38
27.73
28.80
29.11
29.64
31.67
32.64
33.75
34.28
34.70
36.17
36.47
36.57
36.80
39.03
39.66

14. The co-crystal according to claim 11, having an observed experimental X-ray powder diffractogram as shown in FIG. 1.

15. The co-crystal according to claim 11, having a monocline space group P21/n, unit cell dimensions a=15.171 (6) Å, alpha=90°, b=5.9951 (3) Å, beta=92.233(4)°, c=15.995(5) Å, gamma=90° and a volume of 1496.83(11) A³.

16. The co-crystal according to claim 15, having an experimental diffractogram calculated from the single crystal X-ray diffractography as shown in FIG. 2.

17. A pharmaceutical composition comprising the co-crystal according to claim 11 and a pharmaceutically acceptable carrier.

18. A medicament comprising a co-crystal of oxalic acid and 3-benzyl-2-methyl-2,3,3a,4,5,6,7,7a-octahydrobenzo[d]isoxazol-4-one.

19. A method to treat an individual with 3-benzyl-2-methyl-2,3,3a,4,5,6,7,7a-octahydrobenzo[d]isoxazol-4-one, the method comprising
administering to the individual a co-crystal comprising oxalic acid and 3-benzyl-2-methyl-2,3,3a,4,5,6,7,7a-octahydrobenzo[d]isoxazol-4-one.

20. The method of claim 19, wherein the administering is performed in an amount resulting in treatment of a mood disorder, an anxiety disorder, depression, a convulsive state, a learning disability, amnesia, and/or abstinence syndrome from medicaments and drugs.

21. The process according to claim 4, wherein the reacting is conducted with a ratio of solvent volume/mmol of fumaric acid in a range from 2.5 to 5.

22. A method for treatment in an individual of a mood disorder, a disorder of anxiety, depression, a convulsive condition, a learning disability, amnesia, and/or abstinence syndrome from medicaments and drugs, the method comprising
administering an effective amount of co-crystal comprising oxalic acid and 3-benzyl-2-methyl-2,3,3a,4,5,6,7,7a-octahydrobenzo[d]isoxazol-4-one.
